(19) United States
(12) Patent Application Publication
(10) Pub. No.: US 2008/0306384 A1
(43) Pub. Date: Dec. 11, 2008

(54) Apparatus and method for computing 3D ultrasound elasticity images

(75) Inventors: Emad Moussa Boctor, Baltimore, MD (US); Gabor Fichtinger, Kingston (CA); Gregory D. Hager, Baltimore, MD (US); Hassan Rivaz, Baltimore, MD (US)

Correspondence Address: MCKENNA LONG & ALDRIDGE LLP, 1900 K STREET, NW, WASHINGTON, DC, 20006, US

(73) Assignee: The Johns Hopkins University, Baltimore, MD (US)

(21) Appl. No.: 11/905,501

(22) Filed: Oct. 1, 2007

Related U.S. Application Data
(60) Provisional application No. 60/933,888, filed on Jun. 8, 2007.

(51) Int. Cl. A61B 8/00

(52) U.S. Cl. 600/443; 382/128

(57) ABSTRACT

Disclosed is a system and method for computing ultrasound 3D elasticity images. The method includes acquiring ultrasound RF data in a rest state, in which substantially no pressure is applied to a tissue medium, and acquiring ultrasound data in a stressed state, in which pressure is applied to a tissue medium having an aberration, and computing a measured displacement image from the two RF data sets. The method also includes computing an initial estimated displacement image, which is derived from a 3D elasticity model. The method further includes computing an optimization loop, wherein the initial estimated displacement image is adjusted to converge on the measured displacement image. The optimized estimated displacement image is then segmented and superimposed over the rest state ultrasound image. Further, the original 3D elasticity model is adjusted to match the optimized estimated displacement image.

This application claims the benefit of U.S. Provisional Patent Application No. 60/933,888, filed on Jun. 8, 2007, which is hereby incorporated by reference for all purposes as if fully set forth herein.

### BACKGROUND

1. Field of the Disclosure

The present invention generally relates to ultrasound imaging applications. More particularly, the application relates to the use of ultrasound to measure tissue elasticity.

2. Discussion of the Related Art

Ultrasound imaging is commonly used in detecting and targeting tumors, isolating organ structures, and monitoring invasive surgical procedures. One exemplary intraoperative application of ultrasound involves its use in treating tumors. Such treatments include Electron Beam Radiation Therapy (EBRT) and hepatic tumor thermal ablation. A common challenge to these procedures is to accurately image the tumor so that the tumor can be treated most effectively while minimizing damage to the surrounding tissue. A further challenge encountered in such tumor therapies involves the ability to assess the state of the surrounding tissue after treatment or between treatments.

The discussion below pertains to hepatic tumor thermal ablation. However, one will readily appreciate that similar problems and challenges occur in may other ultrasound applications involving imaging a target (e.g., tumor, organ, or ablation) in a surrounding tissue medium.

Conventional brightness (or B-mode) ultrasound has been used for intraoperative target imaging during thermal ablation procedures. However, B-mode ultrasound typically reveals only hyperechoic (i.e., brighter ultrasound signature) areas that result from microbubbles and outgassing from the ablated tissue. The tumor may be isoechoic, meaning that its brightness in ultrasound imagery is substantially indistinguishable from that of the surrounding tissue. In such cases, ablation effectiveness is estimated by the ultrasound-determined position of the ablation probe, and not by imagery of the tumor or surrounding tissue.

Ultrasound elasticity imaging has emerged as an effective technique to mitigate the disadvantages of B-mode ultrasound. Ultrasound elasticity imaging exploits the differences in mechanical properties of the tumor from those of the surrounding tissue medium. By imaging the deformation of the tissue in response to pressure exerted by the ultrasound probe, the contour of the tumor may be extracted from the surrounding tissue. In doing so, the ultrasound system generally tracks the deformation (or strain) of the tissue by tracking the motion of “speckle,” or coherent scattering features within the tissue.

Although an improvement over B-mode ultrasound, related art ultrasound elasticity imaging has limitations. First, related art image processing techniques result in artifacts and noise that degrade the quality of the image, and thus may impede effective target imaging. Second, related art image processing techniques are generally computationally expensive, which often results in significant lag times in image display. The artifacts and noise in related art ultrasound elasticity imagery generally results from speckle decorrelation due to speckle out-of-plane motion, and shadowing.

Another problem regarding related art ultrasound elasticity imaging is that the technician may easily apply too much pressure to the tissue surrounding the tumor. This exacerbates the problem of out-of-plane motion, because the surrounding tissue spreads out of the path (and thus out of the field of view) of the ultrasound probe. Further, applying too much pressure on the surrounding tissue may dislocate the tumor and temporarily alter its shape. Once the pressure is released, the tumor may return to its original location and shape. As such, the location and shape of the imaged tumor (when pressure is applied) may be different from the location and shape of the tumor in its “rest” state. The resulting inaccuracy in target imaging may result in inaccurate delivery of heat or radiation during treatment. Additionally, in the case of multiple treatments, because each technician may apply differing degrees of force, dislocation and distortion of the tumor may further degrade the precision of the determined location and size of the tumor.

Accordingly, what is needed is a system and method for providing ultrasound elasticity imaging that provides higher quality elasticity images more quickly, and in a way that facilitates precise application of pressure.

### SUMMARY OF THE DISCLOSURE

The present invention provides an apparatus and method for computing 3D ultrasound elasticity images that obviates one or more of the aforementioned problems due to the limitations of the related art.

Accordingly, one advantage of the present invention is that it provides for improved target imaging and location of target objects within a tissue medium.

Another advantage of the present invention is that it improves the accuracy of the delivery of treatment of tumors

Another advantage of the present invention is that it improves the quality of ultrasound elasticity images.

Another advantage of the present invention is that it provides for better real time ultrasound elasticity images

Still another advantage of the present invention is that it improves the repeatability of ultrasound elasticity images.

Yet another advantage of the present invention is that it provides better imaging of isoechoic features in a tissue medium.

Additional advantages of the invention will be set forth in the description that follows, and in part will be apparent from the description, or may be learned by practice of the invention. The advantages of the invention will be realized and attained by the structure pointed out in the written description and claims hereof as well as the appended drawings

To achieve these and other advantages, the present invention involves a method for computing an ultrasound displacement image. The method comprises acquiring a first ultrasound image from an ultrasound probe; applying a pressure using the ultrasound probe; acquiring a second ultrasound image from the ultrasound probe; computing a plurality of elasticity parameters corresponding to the first ultrasound image and the second ultrasound image; computing an initial estimated plurality of elasticity parameters, wherein the estimated displacement image corresponds a model; and computing an optimal estimated plurality of elasticity parameters corresponding to the plurality of elasticity parameters and the initial estimated plurality of elasticity parameters.

In another aspect of the present invention, the aforementioned and other advantages are achieved by a method for computing an ultrasound displacement image, which comprises acquiring a first ultrasound image from an ultrasound probe; applying a pressure using the ultrasound probe; acquiring a second ultrasound image from the ultrasound probe; for each sample in one of the first ultrasound image and the second ultrasound image, computing a plurality of displacements corresponding to the first ultrasound image and the second ultrasound image; computing a cost function corresponding to the plurality of displacements; and selecting the displacement from within the plurality of displacements that corresponds to a minimum cost.

In another aspect of the present invention, the aforementioned and other advantages are achieved by a computer readable medium encoded with a program for computing an ultrasound displacement image corresponding to a first ultrasound image and a second ultrasound image. The program comprises for each sample in one of first ultrasound image and the second ultrasound image, computing a plurality of displacements corresponding to the first ultrasound image and the second ultrasound image; computing a cost function corresponding to the plurality of displacements; and selecting the displacement from within the plurality of displacements that corresponds to a minimum cost.

In another aspect of the present invention, the aforementioned and other advantages are achieved by an ultrasound imaging system, which comprises an ultrasound probe; and a computer coupled to the ultrasound probe, wherein the computer has a storage medium encoded with a program for acquiring a first ultrasound image from an ultrasound probe, wherein the first ultrasound image corresponds to a first pressure; acquiring a second ultrasound image from the ultrasound probe, wherein the second ultrasound image corresponds to a second pressure; computing a plurality of elasticity parameters corresponding to the first ultrasound image and the second ultrasound image; computing an initial estimated plurality of elasticity parameters, wherein the estimated displacement image corresponds a model; and computing an optimal estimated plurality of elasticity parameters corresponding to the plurality of elasticity parameters and the initial estimated plurality of elasticity parameters.

In another aspect of the present invention, the aforementioned and other advantages are achieved by an ultrasound probe handle, which comprises a base configured to have an ultrasound probe coupled to it, wherein the base is configured to control an amplitude and frequency of a palpating motion of the ultrasound probe relative to the base.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1 illustrates an exemplary system for processing 3D ultrasound elasticity images;

FIG. 2 illustrates an exemplary process for processing ultrasound elasticity images;

FIGS. 3A-3E depict tissue strain in response to pressure exerted by an ultrasound probe;

FIGS. 4A-C illustrates a cost minimization approach to computing 1-D displacement;

FIG. 4D illustrates steps in an exemplary level-crossing data filtering subprocess to the cost minimization approach;

FIG. 5A illustrates an exemplary “roller coaster” position control handle;

FIG. 5B illustrates two ultrasound fields of view, and their overlapping regions, as an ultrasound probe is rotated and translated using the roller coaster position control handle;

FIG. 6A illustrates an exemplary actuated palpation controller with an installed ultrasound probe; and

FIG. 6B illustrates two overlapping fields of view of an ultrasound probe being controlled using the actuated palpation controller of FIG. 8A.

### DETAILED DESCRIPTION

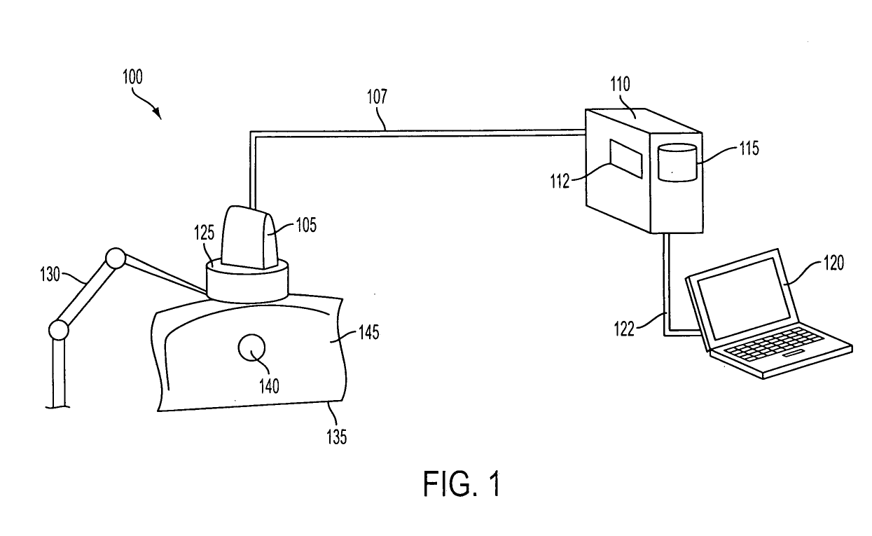

FIG. 1 illustrates an exemplary system 100 for computing 3D ultrasound elasticity images. System 100 includes an ultrasound probe 105, which communicates with a computer 110 over a signal cable 107. Computer 110 may have a processor 112 and a memory 115. Computer 100 may also have a user interface 120, which may be integrated into computer 120, or may be a separate computer that communicates with computer 110 over a network connection 122.

System 100 may also include an optional ultrasound probe mount 125, which may be connected to a mechanical arm 130. Mechanical arm 130, which is optional, may be a robotic arm that is controlled by computer 110, or a passive arm that serves to stabilize probe mount 125. In the latter case, ultrasound probe 105 and probe mount 125 may be moved (translated and rotated) manually by a technician.

Ultrasound probe 105 may be a commercially available ultrasound probe. And ultrasound probe 105, computer 110, and user interface 120 may be components of a commercially available ultrasound imaging system.

Computer 110 may be a single computer or may be multiple computers that may be co-located, or may be remotely located from each other and connected to each other over a network. Similarly, processor 112 may be a single computer processor or multiple processors, which may be distributed over multiple computers.

Memory 115 may include one or more electronic storage media (e.g., hard drive, flash drive, RAM, optical storage, etc.) that may be located within computer 110, or distributed over multiple computers. One skilled in the art will readily appreciate that may such variations to system 100 are possible and within the scope of the disclosure.

Memory 115 may be encoded with computer readable instructions and data (hereinafter “the software”) for performing processes associated with the disclosure. If ultrasound probe 105, computer 110, and user interface 120 are parts of an integrated commercially available ultrasound imaging system, then the software may be installed and integrated into existing machine readable instructions and data that come bundled with the ultrasound imaging system.

FIG. 1 illustrates ultrasound probe 105 acoustically coupled to a patient's anatomy 135, which includes a tissue medium 145. Within tissue medium is an aberration 140. Aberration 140 may be any region or object within tissue medium 140 that has mechanical properties, such as Young's Modulus, that is different from that of surrounding tissue medium 145. Examples of aberration 140 include a tumor, a region of ablated tissue, a foreign object, a cavity resulting from a removed tumor, an organ—such as a prostate gland, and the like. Tissue medium 145 may include a liver, a breast, or any tissue region that surrounds aberration 140.

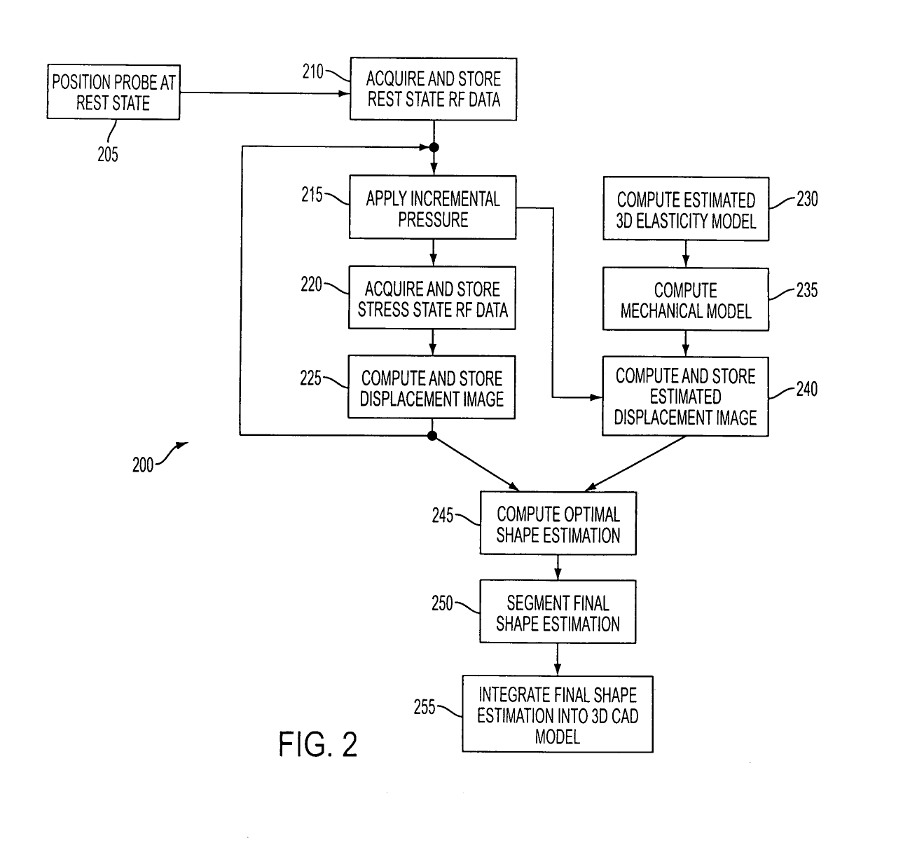

FIG. 2 illustrates an exemplary process 200 for computing 3D ultrasound elasticity images. Process 200 may be implemented by the software stored on memory 115 and executed by processor 112 in conjunction with an ultrasound technician operating system 100.

At step 205, the ultrasound technician may place ultrasound probe 105 against patient's anatomy 135 so that the two are acoustically coupled. This may be done so that pressure sufficient to maintain acoustic coupling is exerted. This initial position of ultrasound probe 105, and the pressure it exerts on patient's anatomy 135, may be referred to as the rest state.

If probe mount 125 is used in conjunction with mechanical arm 130, the technician may establish acoustic coupling between ultrasound probe 105 and patient's anatomy 135 by controlling mechanical arm 130, either manually or by a computer control via user interface 120.

At step 210, processor 112 executes instructions to acquire RF data from ultrasound probe 105 while ultrasound probe 105 is in the rest state. As used herein, RF data may refer to the image data acquired by ultrasound probe 105, which may include a plurality of RF lines that make up a two dimensional ultrasound image frame. Each RF line may be a plurality of echo samples detected by ultrasound probe 105 along a single detector field of view. In other words, an RF line may be a series of samples corresponding to retrieved echoes along a single ID profile projected from ultrasound probe 105. Further to step 210, processor 112 executes instructions to store the RF data in memory 115 as rest state RF data.

At step 215, ultrasound probe 105 may be manipulated to apply an increment of pressure on patient's anatomy 135. In doing so, the ultrasound technician may manually apply pressure on ultrasound probe 105 along a direction substantially toward aberration 140. If mechanical arm 130 is used, the ultrasound technician may apply pressure by manually or electronically controlling manual arm 130.

As used herein, an increment of pressure may refer to a sufficient amount of pressure to cause measurable displacement of tissue medium 145 and aberration 140 without causing speckle in tissue medium 145 to move out of the image plane of ultrasound probe 105 and lead to image decorrelation. Further, an increment of pressure may be limited so that the shape and position of aberration 140 may remain somewhat constant, and not be overly distorted by pressure exerted by ultrasound probe 105.

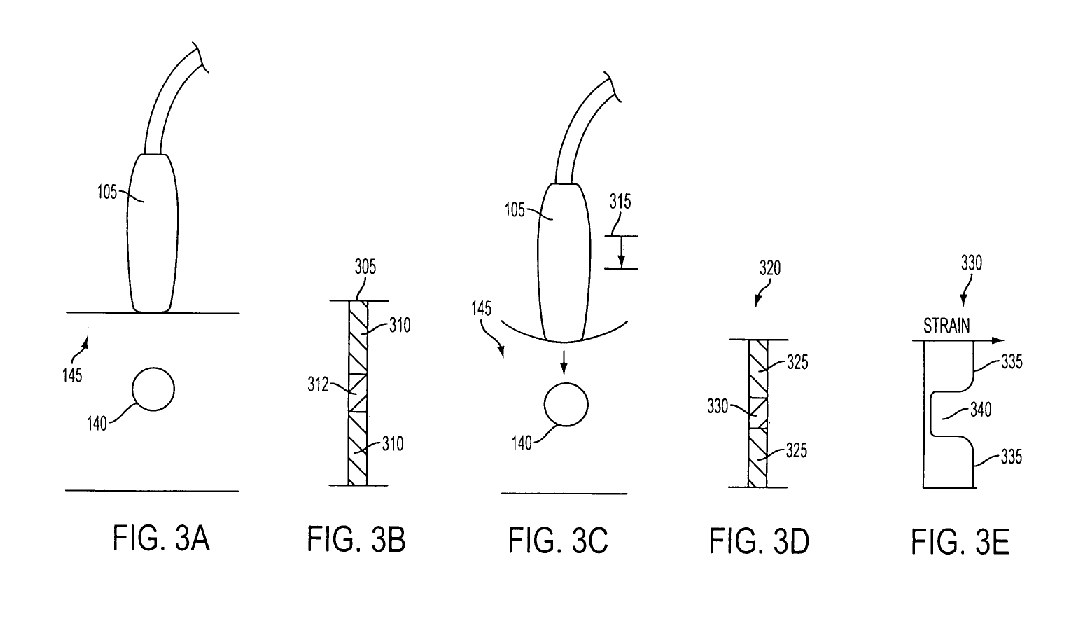

FIGS. 3A-3E graphically depict the rest state and the stress state, and the resulting displacement and strain of tissue medium 145 and aberration 140.

FIG. 3A illustrates ultrasound probe 105 in the rest state, while minimal pressure is exerted on tissue medium 145 and aberration 140.

FIG. 3B graphically illustrates a rest state exemplary RF line 305, including the tissue medium rest ultrasound signature 310 and the aberration rest ultrasound signature 312.

FIG. 3C illustrates ultrasound probe 105 in the stress state, in which ultrasound probe 105 has translated substantially toward aberration 140 by a probe translation distance 315. Also illustrated in FIG. 3C is the resulting displacement of aberration stress state ultrasound signature 330 relative to aberration rest state ultrasound signature 312.

FIG. 3D graphically illustrates a stress state RF line 320, in which tissue medium stress state ultrasound signature 325 changes from the corresponding rest state ultrasound signature 310 in response to the pressure exerted by ultrasound probe 105. Tissue medium stress state 325 may respond to the pressure in such a way that ultrasound probe 105 may detect a compression of speckle within tissue medium 145. This is graphically depicted by the compression of parallel lines within tissue medium stress state ultrasound signature 325 relative to tissue medium rest state ultrasound signature 310.

FIG. 3E illustrates an exemplary strain profile 330, which graphically depicts tissue medium strain 335, which is greater than aberration strain 340.

One skilled in the are will recognize that the amount of pressure referred to by the term increment of pressure will vary, depending on the location of aberration 140 and tissue medium 145 within patient's anatomy 135. For example, if aberration 140 is a prostate gland, more pressure will have to be exerted by ultrasound probe 105 to cause measurable displacement of tissue medium 145 because of intervening anatomical features, such as the bladder. In contrast, less pressure will be exerted in the case where tissue medium 145 is breast tissue and aberration 140 is a tumor.

With pressure exerted by ultrasound probe 105, the position of ultrasound probe 105, and the resulting displacement of tissue medium 145 and aberration 140, may be referred to as the stress state.

At step 220, processor 112 executes instructions to acquire RF data from ultrasound probe 105 while ultrasound probe 105 is in the stress state. Processor 112 further executes the software to store the RF data in memory 115 as stress state RF data.

At step 225, processor 112 executes instructions to compute a displacement image using the rest state RF data and the stress state RF data. In doing so, processor 112 executes instructions to retrieve the rest state RF data and the stress state RF data from memory 115. Then, processor 112 may execute instructions to compute the displacement between the rest state RF data and the stress state RF data. Displacement may refer to change in location of a given point (within tissue medium 145 or within aberration 140) between the rest state and the stress state, wherein the given point is present within both the rest state RF data and the stress state RF data. The given point must be commonly present in the rest state RF data and the stress state RF data with a sufficiently high degree of correlation to be identified uniquely in both sets of RF data. Computing a displacement image may be done by one of at least two ways. First, the software may include instructions for computing a displacement image using window correlation techniques that are known to the art. Alternatively, the software may employ a dynamic programming approach, which would include instructions for computing a minimum cost-based displacement image as described below.

In calculating a minimum cost displacement image, processor 112 may execute instructions to compute a cost function for each sample within the rest state and stress state RF data sets. It may do so according to the following relation:

$$C ( i , {d}_{i} ) = {min}_{{d}_{i - 1}} \{ C ( i - 1 , {d}_{i - 1} ) + wZ ( {d}_{i} , {d}_{i - 1} ) \} + Δ ( i , {d}_{i} )$$

where i is the i^{th} sample within either the rest state of the stress state RF data; and d is the displacement of the i^{th} sample between the rest state RF data and the stress state RF data, where d may be bounded by a maximum magnitude displacement range dₘᵢₙ≦d≦dₘₐₓ. For example, the maximum magnitude displacement range may be set to one sample in distance, in which case dᵢ₋₁ may be limited to either dᵢ−1, dᵢ, or dᵢ+1. Limiting the search range of the displacement may greatly reduce the computational expense of computing the displacement image because it greatly reduces the range of values for which displacement values are computed.

The term w corresponds to a weighting factor, which is a configurable parameter that may be adjusted to improve the quality of the computed displacement image. Weighting factor w may only need be set once.

The expression Z(dᵢ,dᵢ₋₁) is function corresponding to the smoothness of the displacement, and may be computed according to the relation (dᵢ−dᵢ₋₁)^{k}, where k is a configurable parameter to set the smoothness of the sample-by-sample displacement. For example, setting k to 2 limits large jumps in estimated displacement.

The expression Δ(i,d) corresponds to a sum of the absolute distances between the rest state and the stress state RF data. This may be computed according to the relation Δ(i,d)=|g(i)−g′(i+d)|, where g(i) is the signal amplitude of the i^{th} sample of rest state RF data, and g′(i) is the signal amplitude of the i^{th} sample of the stress state RF data. Alternatively, g(i) and g′(i) may refer to the stress state RF data and the rest state RF data, respectively.

Processor 112 may execute instructions to “memoize” the computed optimum value for dᵢ₋₁ using the following function:

$$M ( i , d ) = {argmin}_{{d}_{i - 1}} \{ C ( i - 1 , {d}_{i - 1} ) + wZ ( {d}_{i} , {d}_{i - 1} ) \} .$$

Processor 112 may execute instructions to compute the cost function C for i=l . . . m, where m is the number of samples in the rest state and stress state RF data sets. The minimum cost at i=m gives the displacement at the ith sample. Processor 112 may then execute the software to trace the minimum cost function back to i=l using the above memoization function M(i,d) to calculate all the displacements D for all of the samples of the rest state and stress state RF data sets according to the following relation:

$$\begin{matrix}D ( i ) = {argmin}_{{d}_{i}} \{ C ( i , {d}_{i} ) \} , i = m \\ D ( i ) = M ( i + 1 , D ( i + 1 ) ) , i = 1 … m - 1.\end{matrix}$$

The above description pertains to a single RF line common to two ultrasound images: the rest state RF data set, and the stress state RF data set. Processor 112 may repeat the above computational steps for each corresponding RF line in the two RF data sets.

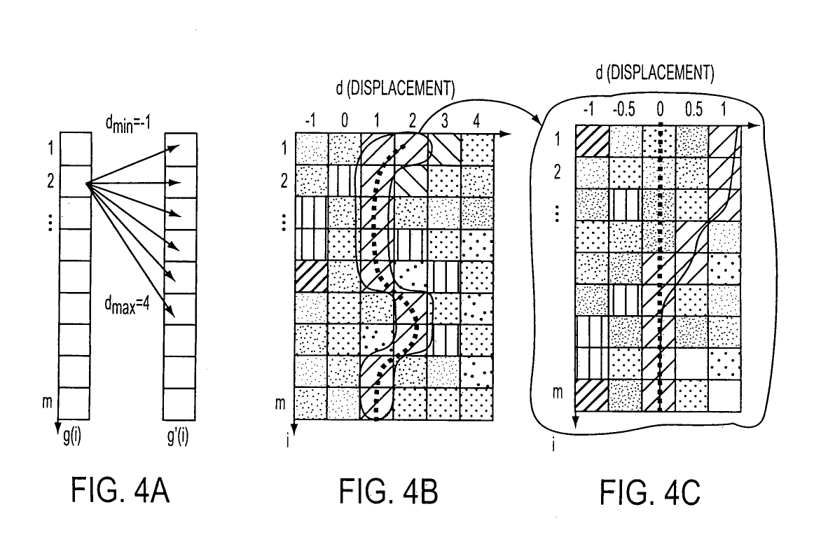

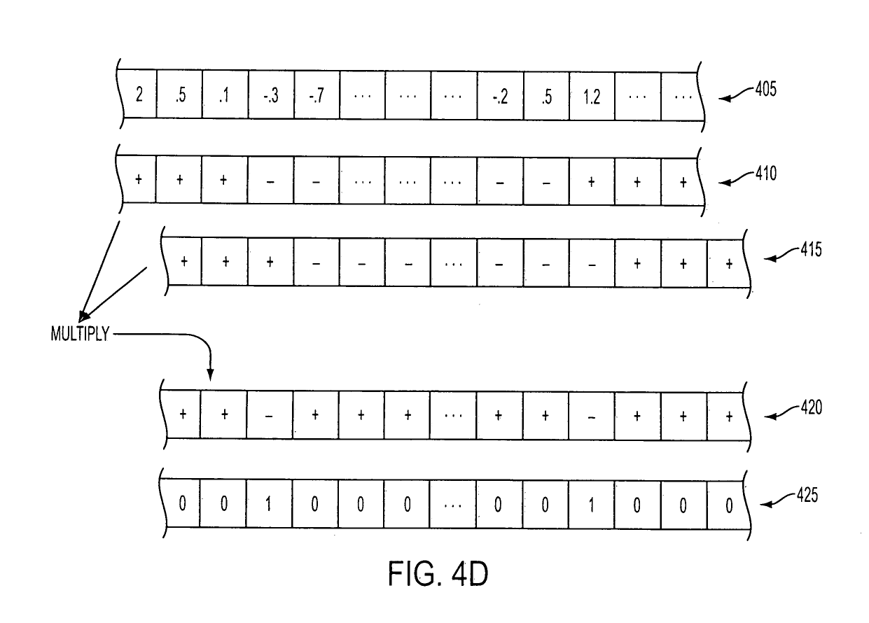

FIGS. 4A-4D illustrate two RF data sets g(i),g′(i) for a given RF line, and how the above-described minimum cost displacement may be computed for a given RF line. RF line g(i) may be the RF line from the rest state data set, and RF line g′(i) may be the RF line of the stress state, or vice versa. FIG. 4A illustrates an example in which dₘᵢₙ is set to −1, and dₘₐₓ is set to +4. One skilled in the art will appreciate that different dₘᵢₙ and dₘₐₓ values may be used in a tradeoff between maximum expected displacement and computational complexity.

The above dynamic programming approach, which computes 1D displacement, may be enhanced so the displacement may be computed, not only within a single RF line, but between RF lines. In this case, 2D displacement may be computed. This may be particularly useful because tissue medium 145 may be displaced in the stress state in more than the axial direction (i.e., toward aberration 140). In this case, displacement within the field of view of ultrasound detector 105, including displacement across RF lines, may be computed. An exemplary process for computing 2D displacement is described below.

In computing displacement in the lateral direction (across n RF lines) as well has in an axial direction (within an RF line), processor 112 may execute instructions to compute the distance between rest state and stress state as follows:

Δ(i,j,dₐ,dₗ)=|gⱼ(i)−g′_{j+d}_{ₗ}(i+dₐ)|

where d_{a,min}≦dₐ≦d_{a,max} and d_{l,min}≦d₁≦d_{l,max} are the axial and lateral displacements, respectively, and j=l . . . n refers to the j^{th} RF line, and i=l . . . m.

In this example, processor 112 may execute instructions to compute smoothness according to the following relation

Z(dₐ_{ᵢ},dₗ_{ᵢ},dₐ_{_{i−1}},dₗ_{_{i−1}})=(dₐ_{ᵢ}−dₐ_{ᵢ})²−(dₗ_{ᵢ}−dₗ_{_{i−1}})²

Processor 112 then executes the software to compute the cost function of the i^{th} sample of the j^{th} RF line according to the following relation

$${C}_{j} ( {d}_{a} , {d}_{l} , i ) = {min}_{{δ}_{a} , {δ}_{l}} \{ \begin{matrix}\frac{\begin{matrix}{C}_{j} ( {δ}_{a} , {δ}_{l} , i - 1 ) + \\ {C}_{j - 1} ( {δ}_{a} , {δ}_{l} , i )\end{matrix}}{2} + \\ wZ ( {d}_{a} , {d}_{l} , {δ}_{a} , {δ}_{l} )\end{matrix} \} + Δ ( {d}_{a} , {d}_{l} , i )$$

where δₐ and δₗ are parameters for minimizing the cost function, which are stored in memory 115 for all dₐ, dₗ and i values. This form of the cost function may allow the computation of the displacement for each RF line using the cost values of the previous RF line. Processor 112 executes instructions to compute and minimize the cost function of the j^{th} line, Cⱼ(dₐ,dₗ,i), resulting in a displacement map, which is stored in memory 115. The cost function Cⱼ(dₐ,dₗ,i) is also used for calculation of the next cost function, Cⱼ₊₁(dₐ,dₗ,i), and may then be deleted from memory. This may make the required amount of space in memory 115 substantially independent of the number of RF lines.

Both of the 1D and 2D displacements described above provide displacement in integer sample resolution. For example, for each sample i, there is a resulting displacement i+D(i), where D(i) is an integer number of samples. The above-described minimum cost computational processes may be enhanced to compute sub-sample resolution displacement. This may be done several ways. For example, the software may include instructions to implement a post-processing statistical method, such as a least squares fitting. However, such an approach may be inordinately computationally expensive.

An alternate approach to computing sub-sample resolution displacement may involve the following. For each minimum cost sample i (as computed above) in the rest state RF data set, processor 112 executes instructions to interpolate multiple sub-samples between sample range [i−1, i+1]. The number of sub-samples to be interpolated may depend on a sampling factor γ, which may be a configurable parameter stored in memory 115. This results in an “up-sampled” interpolated rest state RF data array between i−1 and i+1.

Processor further interpolates the stress state RF data set by the same sub-sample resolution within the range [i +D(i)−1,i+D(i)+1], resulting in an up-sampled interpolated stress state RF data array.

Processor 112 may then execute instructions to run one of the above-described minimum cost computational procedures on the up-sampled interpolated rest state RF data array (within the range [i−1,i+1]), and the up-sampled stress state RF data array (within the range [i+D(i)−1,i+D(i)+1]), resulting in an interpolated sub-sample resolution displacement. Processor 112 may then execute instructions to repeat this for all of the minimum cost displacements computed above, and then store the resulting sub-sample minimum cost displacements in memory 115.

FIG. 4C illustrates exemplary results of a sub-sample displacement estimation corresponding to the integer sample displacement computation illustrated in FIG. 4B.

Variations to the above dynamic programming minimum cost-based are possible and within the scope of the disclosure. For example, it may be the case that not all of the samples within the rest state and stress state RF data sets need be used to accurately compute a displacement image at step 225. For example, it may be that an adequate displacement image may be created with as few as 20% of the samples within the rest state and the stress state RF data sets. One way of extracting a pertinent subset of the full RF data sets is to compute a “level crossing” data array, wherein the rest state RF data set may have a corresponding rest state level crossing data array, and the stress state RF data set may have a corresponding stress state level crossing data array.

FIG. 4D illustrates steps of an exemplary process for computing a level crossing data array, in which the level corresponds to a zero crossing. Processor 112 then executes instructions to retrieve an RF data set 405 (rest state or stress state) from memory 115. Processor 112 executes instructions to create a sign data array 410 containing the signs of each corresponding value of RF data set 405. Then processor 112 executes instructions to create a shifted sign data array 415, which is a copy of sign data array 410 that is shifted by one sample. Processor 112 then executes instructions to multiply sign data array 410 and shifted sign data array 415, creating a sign product data array 420, which may be further represented as a binary zero crossing data array 425. In binary zero crossing data array 425, all “1” values correspond to a zero crossing of the corresponding RF data set.

Having computed a binary zero crossing data array 425 for both the rest state and stress state RF data sets, processor 112 may execute instructions to compute either of the above-described minimum cost processes, using only the “1” values of the respective binary zero crossing data arrays as inputs.

By using only the zero crossing samples of the RF data sets, a displacement image may be computed in a way that is computationally much less expensive, while providing a displacement image having sufficient accuracy for the purposes of process 200.

The above exemplary process for computing a level crossing data array corresponds to a zero crossing example. However, one skilled in the art will recognize that an offset may be added to the RF data sets to provide a binary level crossing data array corresponding to crossings of a predetermined signal level other than zero. Further, this process may be expanded to include more than one level crossing. For example, two RF data signal levels (e.g., −1 Volt and +1 Volt) may be used. This may result in approximately twice as many level crossings as a single level crossing. This may increase the resolution of the resulting displacement image, while still gaining the benefits of reduced computational expense. It will be apparent to one skilled in the art that such variations are possible and within the scope of the disclosure.

Accordingly, regardless of which of the above stated approaches is used, the result of step 225 is a displacement image, which is stored in memory 115.

As illustrated in FIG. 2, process 200 may return to step 215, in which another increment of pressure is exerted by ultrasound probe 105 on the patient's anatomy 135. Steps 215-225 may then be repeated. In repeating steps 215-225, a plurality of incremental displacement images may be computed and stored. In computing the displacement images in subsequent iterations of step 225, displacement may be computed relative to two successive pressure increments, or displacement may be computed relative to the most recent stress state RF data set and the original rest state RF data set. Further, depending on the frame rate of ultrasound system of system 100, steps 215-225 may run repeatedly as ultrasound probe 105 is palpated, or moved axially in an oscillatory motion.

Returning to process 200, at step 230, processor 112 my execute instructions to compute an estimated 3D elasticity model of aberration 140. In doing so, a physician may estimate the location, shape, and elasticity of aberration 140, and enter this information into computer 110 user interface 120. Processor 112 may then execute the software to convert this information into a 3D elasticity model of aberration 140 within surrounding tissue medium 145, and store the 3D elasticity model in memory 115. One skilled in the art will readily appreciate that numerous computational techniques may be employed to represent aberration 140 in a 3D space, all of which are within the scope of the disclosure.

At step 235, processor 112 may execute instructions to compute a mechanical model, which converts the 3D elasticity model into a format from which the estimated displacement image may be derived at step 240. Various mechanical models may be used. Exemplary mechanical models include a finite element model, and a boundary element model. In the case of a finite element model, the resulting finite element model may include a plurality of elements, each of which has location, a dimension, and an elasticity. The finite element model may be a 2D model, which may be a “slice” of the 3D elasticity model of step 230, wherein the “slice” corresponds to the field of view of ultrasound probe 105. Numerous finite element model techniques are known to the art, and one skilled in the art will recognize that many such models may be used here within the scope of the disclosure.

By using a finite element model, wherein each element has an isotropic elasticity, it is possible to model the elasticity of aberration 140 in the presence of tissue medium 145 by applying a linear model between neighboring elements to compute a displacement image. One such linear elasticity model is Navier's equation:

$$ρ \frac{{\partial }^{2} u}{\partial {t}^{2}} - ∇ \cdot c ∇ u = K$$

where ρ is the material density, K are the body forces, and c is a tensor, wherein each entry is a function of G (shear modulus), E (Young's modulus), and ν (Poisson's ratio). It will be readily apparent to one skilled in the art that there are numerous ways of coding an implementation of Navier's equations, each of which are within the scope of the disclosure. Further, one skilled in the art will recognize that other numerical modeling techniques may be employed within the scope of the disclosure.

For example, instead of implementing a finite element model, processor 112 may execute instructions to implement a boundary element model. A boundary element model is a numerical computational method of solving linear partial differential equations that have been formulated as integral equations. In implementing a boundary element model, processor 112 executes instructions to use predetermined boundary conditions to fit boundary values into an integral equation, rather than values throughout the space defined by a partial differential equation. With this done, processor 112 may execute instructions to use the integral equation to compute the displacement at any desired point in the interior of the solution domain (i.e., the estimated displacement image). Boundary element models are computationally less expensive than finite element models. For further detail regarding boundary elements models, one may refer to Transformation of Domain Effects to the Boundary (Advances in Boundary Elements,) (Hardcover) by Youssef F. Rashed (Editor), and Mitic P, Rashedb Y F, “Convergence and stability of the method of meshless fundamental solutions using an array of randomly distributed sources,” Engineering Analysis with Boundary Elements, Volume 28, Issue 2, February 2004, Pages 143-153.

In computing a boundary element model, a physician or technician may select estimated boundary points of aberration 140 via user interface 120. Processor 112 may then execute instructions to store these boundary points in memory 115.

At step 240, processor 112 executes instructions to compute an initial estimated displacement image. In doing so, processor 112 may retrieve the model generated at step 235, and may retrieve information regarding the incremental pressure applied at step 215, may compute an estimated displacement image and store the estimated displacement image in memory 115.

At step 245, processor 112 executes instructions to compute an optimal shape estimation corresponding to aberration 140. In doing so, processor 112 may execute instructions to iteratively adjust the estimated displacement image computed at step 240 until it fits the displacement image computed at step 225. Once the estimated displacement field of step 240 is sufficiently similar to the measured displacement field of step 225, the deformed estimated displacement field may yield the contours of aberration 140. In doing so, processor 112 may iterate the following objective function:

$$\hat{S} = argmin \{ 𝒥 ( S ) = {\sum }_{i = 1}^{N} {\sum }_{j = 1}^{M} W ( i , j ) ‖ \hat{u} ( i , j ) - u ( i , j ; S ) ‖ \}$$

where Ŝ are the estimated shape parameters; û is the displacement computed at step 225; u is the estimated displacement computed at step 240; [symbol] (S) is the objective function; M is the number of samples in a single RF line; N is the number of RF lines; i and j are indices into the 2D image; and W is a correlation map, which serves as a weighing function to minimize the effects of lower quality displacements computed at step 225.

The shape parameters S and Ŝ may include the location of aberration 140 in 2D space, the size of aberration 140, and the orientation of aberration 140.

By using the correlation map W, it is possible to estimate the shape parameters Ŝ for aberration 140 even if the displacement image computed at step 225 has incomplete information. For example, it may be the case that the displacement image computed at step 225 only provides an image of a “bump” in tissue medium 145 concealing aberration 140. In this case, by exploiting the best displacement data points (i.e., correlation map W) an estimation of the shape parameters Ŝ may be computed even with incomplete ultrasound imagery of aberration 140.

If steps 215-225 are iterated multiple times, as described above, then the expression for Ŝ may have an additional summation term. The additional summation may be for multiple displacement images computed in the multiple iterations of step 225. Accordingly, in addition to the M samples within an RF line, and the N RF lines within an image, there may be an additional summation for the number of images. This may improve the fidelity of the computed shape parameters Ŝ because there would be opportunity to integrate more displacement estimations that have relatively high correlations, and thus higher weighing factors W.

As mentioned above, the objective function for Ŝ is iterated until the objective function is minimized to within a tolerance, which may be a parameter stored in memory 115. Once completed, processor 112 may store the resulting shape parameters Ŝ in memory 115. Accordingly, as used herein, “optimal” may refer to an optimization numerical computation technique, and does not necessary mean that the estimated displacement image computed at step 240 has to perfectly match the displacement image computed at step 225.

At step 250, processor 112 executes instructions to segment the shape estimation computed at step 245. In doing so, processor 112 may retrieve the rest state RF data acquired at step 210, and superimpose the estimated shape within the image corresponding to the rest state RF data. In doing so, aberration 140 may be seen clearly in the surrounding tissue medium 145.

At step 255, processor 112 may execute instructions to integrate the segmented image computed at step 250 into a 3D image space, such as a 3D CAD model. In doing so, processor 112 may execute instructions to generate a 3D CAD model in the coordinate space corresponding to the estimated 3D elasticity model of step 230. Further, system 100 may include a commercially available ultrasound tracking and registration system (not shown), which provides a location and orientation of ultrasound probe 105 in a 3D space referenced to an external reference frame. One skilled in the art will recognize how to incorporate data from an ultrasound tracking and registration system into system 100, and to use the tracking data to register the segmented image computed at step 250 into a 3D image space.

Further to step 255, processor 112 may execute instructions to transform the optimal shape estimation of step 245 and “back out” the mechanical model of step 235 to compute an optimal 3D elasticity model, which is the optimized version of the estimated 3D elasticity model generated at step 230. Processor 112 may execute instructions to store the optimized 3D elasticity model in memory 115, and may display the optimized 3D elasticity model on the screen of user interface 120.

Process 200 may be performed to generate a segmented image, in which aberration 140 is visible, including cases in which aberration is only partly visible, as stated above. Process 200 may be repeated multiple times, each time with ultrasound probe 105 in a different location and orientation, so that a 3D model of aberration 140 may be assembled.

Variations to process 200 are possible. For example, as described in the example above, steps 225 and 240 involve computing and storing displacement images, and step 245 involves optimizing the estimated displacement image of step 240 by comparing it to the displacement image of step 225. However, process 200 is not limited to displacement images, and may by applied to other elasticity parameters. For example, steps 225 may involve extracting strain data from the rest state and stress state RF data sets; and step 240 may involve deriving strain data from the 3D elasticity module. Further, instead of strain, these steps may respectively involve deriving Poisson's Ratio. In either of these examples, the optimization step of 245 computes an optimized version of the estimated parameter set from step 240. One skilled in the art will readily appreciate that such variations are within the scope of the disclosure.

As described above, system 100 may include an ultrasound probe mount 125 and a mechanical arm 130. In many applications, it may be impractical to use an ultrasound probe 105 that is mounted to a mechanical arm. Further, as discussed above, the fidelity of ultrasound elasticity images may depend on well controlled and repeatable applications of incremental force at step 215.

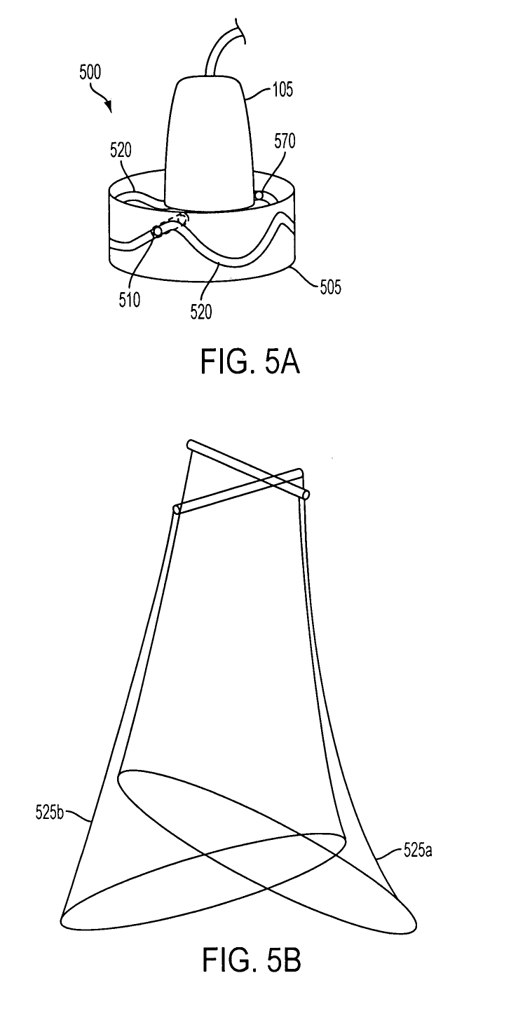

FIG. 5A illustrates an exemplary ultrasound probe handle 500, which may be used to apply controlled and repeatable palpation by ultrasound probe 105 on patient's anatomy 135. Probe handle 500 may include a base 505, in which a commercially available ultrasound probe 105 may be mounted. Affixed to ultrasound probe 105 may be a plurality of guide pins 510, which engage an oscillatory groove 520 disposed within an inner surface of base 505.

In using probe handle 500, a technician may position ultrasound probe 105 so that pins 510 are substantially at a peak position within oscillatory groove 520. Then the technician may place probe handle against patient's anatomy 135. With ultrasound probe 105 turned on so that it is acquiring image data, the technician may rotate ultrasound probe 105 within probe handle 500. In doing so, oscillatory groove 520 may guide the position and orientation of ultrasound probe 105 so that tissue medium 145 and aberration 140 are displaced in a way that out-of-plane motion of speckle is minimized, distortion and dislocation of aberration 140 is minimized, and the pressure and displacement are done in a repeatable manner.

FIG. 5B illustrates two ultrasound fields of view 525a and 525b, which result from ultrasound probe 105 being rotated within probe handle 500. As illustrated, given the divergence of the field of view of ultrasound probe 105, successive frames of ultrasound data may have a large extent of overlap. This not only may provide for good correlation between rest state and stressed state RF data (as discussed with regard to process 200), but in also may provide for known relative orientations of image planes for integrating multiple segmented images into a 3D CAD model (as discussed with regard to step 255 of process 200)

Probe handle 500 may have a variety of bases 505 having different oscillatory grooves 520. For example, as described above, ultrasound elasticity imaging of a prostate may require greater displacement than imaging of a breast tumor. Accordingly, different bases 505 may be provided having different amplitudes and/or frequencies of oscillation.

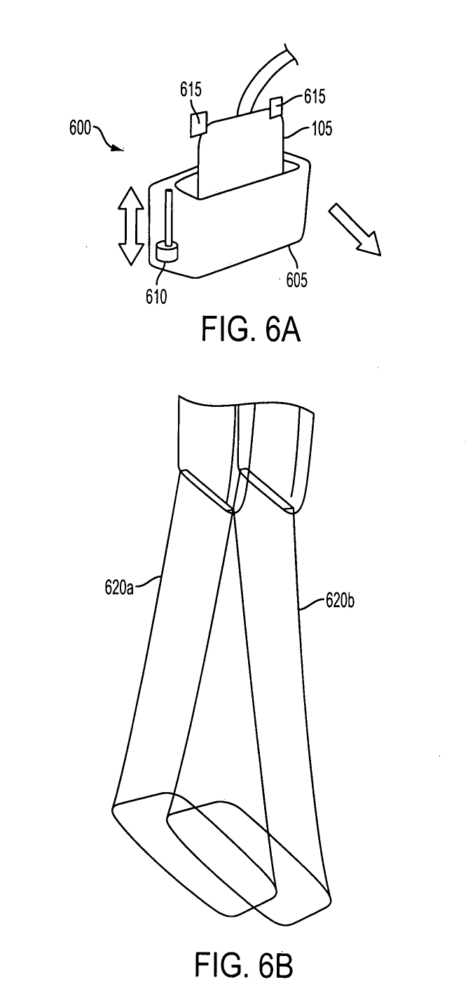

FIG. 6A illustrates another exemplary ultrasound probe handle 600. Probe handle 600 may provide for controlled and repeatable application of pressure and displacement by ultrasound probe 105 against patient's anatomy 135. Probe handle may include a base 605 into which ultrasound probe 105 is mounted, and one or more actuators 610 that translate ultrasound probe 105 substantially along ultrasound probe 105 image plane. Probe handle 600 may also include at least one fiducial marker 615, which may work in conjunction with a commercially available optical tracking system.

Probe handle 600 may function as follows. The ultrasound technician may place probe handle 600 against the patient's anatomy 135, and then engage actuators 610. Actuators 610 may palpate ultrasound probe 105 in an oscillatory motion, similar to the amplitude of the motion induced by the oscillatory grooves 520 of probe handle 500. In this manner, controlled and repeatable pressure and displacement may be exerted on tissue medium 145 and aberration 140.

Depending on the target aberration (e.g., prostate or breast tumor), the extent of motion by actuators may be adjusted by a controller (not shown), which may include motor control software running on computer 110.

In order to perform a 3D scan of tissue medium 145 and aberration 140, it may be necessary to translate probe handle 600. In translating probe handle 600, it may be required that the spatial frequencies of the oscillatory motions induced by actuators 610 be constant. In other words, palpation induced by the actuators may need to have the same number of oscillations per a given translational distance over patient's anatomy 135. To accomplish this, an optical tracking system (not shown) may detect the position and velocity of ultrasound probe 105 via fiducial markers 615. The optical tracking system may provide the position and velocity data to computer 110. Processor 112 may execute instructions to control actuators 610 to adjust the frequency of palpation so that it is proportional to the translational speed of probe handle 600 induced by the ultrasound technician. As such, if different ultrasound technicians translate probe handle 600 at different speeds, then actuators 610 may be controlled to compensate for theses differences and provide for substantially consistent frequency of palpation as a function of linear distance.

FIG. 6B illustrates two ultrasound probe fields of view 625a and 625b. As illustrated, depending on the frame rage of ultrasound probe 105, and the speed of translational motion of probe handle 600, there may be considerable overlap between the fields of view of successive ultrasound image frames. Further, it has been determined that speckle features may have dimensions that are several times the thickness of field of view 625a or 625b. Accordingly, by controlling the frequency of palpation, a sufficient number of RF data sets may be acquired over a given volume to provide elasticity images of a 3D volume.

Variations to probe handle 600 are possible. For example, fiducial markers 615 may be disposed on base 605, instead of (or in addition to) ultrasound probe 105. This may obviate the need to attach fiducial markers to ultrasound probe 105.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

What is claimed is:

1. A method for computing an ultrasound displacement image, comprising:
acquiring a first ultrasound image from an ultrasound probe;
applying a pressure;
acquiring a second ultrasound image from the ultrasound probe;
computing a plurality of elasticity parameters corresponding to the first ultrasound image and the second ultrasound image;
computing an initial estimated plurality of elasticity parameters, wherein the estimated displacement image corresponds to a model; and
computing an optimal estimated plurality of elasticity parameters corresponding to the plurality of elasticity parameters and the initial estimated plurality of elasticity parameters.

2. The method of claim 1, wherein the plurality of elasticity parameters includes a displacement image, the initial estimated plurality of elasticity parameters includes an initial estimated displacement image, and the optimal estimated plurality of elasticity parameters includes an optimal estimated displacement image.

3. The method of claim 2, further comprising segmenting the optimal estimated displacement image.

4. The method of claim 3, further comprising integrating the optimal estimated displacement image into a 3D CAD model.

5. A method for computing an ultrasound displacement image, comprising:
acquiring a first ultrasound image from an ultrasound probe;
applying a pressure using the ultrasound probe;
acquiring a second ultrasound image from the ultrasound probe;
for each sample in one of the first ultrasound image and the second ultrasound image, computing a plurality of displacements corresponding to the first ultrasound image and the second ultrasound image;
computing a cost function corresponding to the plurality of displacements; and
selecting the displacement from within the plurality of displacements that corresponds to a minimum cost.

6. The method of claim 5, wherein computing the plurality of displacements comprises computing the displacement within a maximum magnitude displacement range.

7. The method of claim 6, wherein computing the cost function comprises:
computing a smoothness corresponding to the displacement; and
computing a sum of the absolute distances between the first ultrasound image and the second ultrasound image.

8. The method of claim 5, further comprising:
interpolating the first ultrasound image for a first plurality of sub-samples over a first input sub-sample range;
interpolating the second ultrasound image for a second plurality of sub-samples over a second input sub-sample range; and
computing a second cost function corresponding to the first plurality of sub-samples and the second plurality of sub-samples.

9. The method of claim 8, wherein the first input sub-sample range is [i−1,i+1], wherein i corresponds to a sample in the first ultrasound image; and the second input sub-sample range is [i+D(i)−1,i+D(i)+1], wherein D(i) corresponds to the displacement.

10. A computer readable medium encoded with a program for computing an ultrasound displacement image corresponding to a first ultrasound image and a second ultrasound image, the program comprising:
for each sample in one of first ultrasound image and the second ultrasound image, computing a plurality of displacements corresponding to the first ultrasound image and the second ultrasound image;
computing a cost function corresponding to the plurality of displacements; and
selecting the displacement from within the plurality of displacements that corresponds to a minimum cost.

11. The computer readable medium of claim 10, wherein computing the plurality of displacements comprises computing a displacement within a maximum magnitude displacement range.

12. The computer readable medium of claim 11, wherein computing the cost function comprises:
computing a smoothness corresponding to the displacement; and
computing a sum of the absolute distances between the first ultrasound image and the second ultrasound image.

13. The computer readable medium of claim 10, wherein computing the plurality of displacements comprises computing an axial displacement.

14. The computer readable medium of claim 13, wherein computing the plurality of displacements further comprises computing a lateral displacement.

15. The computer readable medium of claim 10, further comprising:
computing a first level crossing data array corresponding to the first ultrasound image; and
computing a second level crossing data array corresponding to the second ultrasound image, before computing the plurality of displacements.

16. An ultrasound imaging system, comprising:
an ultrasound probe; and
a computer coupled to the ultrasound probe, wherein the computer has a storage medium encoded with a program for acquiring a first ultrasound image from an ultrasound probe, wherein the first ultrasound image corresponds to a first pressure; acquiring a second ultrasound image from the ultrasound probe, wherein the second ultrasound image corresponds to a second pressure; computing a plurality of elasticity parameters corresponding to the first ultrasound image and the second ultrasound image; computing an initial estimated plurality of elasticity parameters, wherein the estimated displacement image corresponds a model; and computing an optimal estimated plurality of elasticity parameters corresponding to the plurality of elasticity parameters and the initial estimated plurality of elasticity parameters.

17. The system of claim 16, wherein the plurality of elasticity parameters includes a displacement image, the initial estimated plurality of elasticity parameters includes an initial estimated displacement image, and the optimal estimated plurality of elasticity parameters includes an optimal estimated displacement image.

18. The system of claim 17, wherein the computer readable medium is further encoded with a program for segmenting the optimal estimated displacement image.

19. The system of claim 18, wherein the computer readable medium is further encoded with a program for integrating the optimal estimated displacement image into a 3D CAD model.

20. An ultrasound probe handle, comprising a base configured to have an ultrasound probe coupled to it, wherein the base is configured to control an amplitude and frequency of a palpating motion of the ultrasound probe relative to the base.

21. The ultrasound probe handle of claim 20, wherein the base comprises an actuator that translates the ultrasound probe substantially along an plane parallel to an image plane of the ultrasound probe.

22. The ultrasound probe handle of claim 21, wherein the base comprises a fiducial marker.

23. The ultrasound probe handle of claim 22, further comprising:
an optical tracking system; and
a computer coupled to the optical tracking system and the actuator.

24. The ultrasound probe handle of claim 23, wherein the computer comprises a computer readable medium encoded with a program for measuring a translational velocity of the ultrasound probe, and controlling a palpation frequency of the actuator, wherein the palpation frequency is proportional to the translational velocity.

25. The ultrasound probe of claim 20, wherein the base has an oscillatory groove, wherein the oscillatory groove is configured to engage a guide pin, wherein the guide pin is coupled to the ultrasound probe.
